(19) United States
(12) Patent Application Publication
(10) Pub. No.: US 2020/0353270 A1
(43) Pub. Date: Nov. 12, 2020

(54) COMMUNICATION SYSTEM OF IMPLANTABLE DEVICE

(71) Applicant: HANGZHOU NANOCHAP ELECTRONICS CO., LTD., Hangzhou (CN)

(72) Inventor: Jiawei YANG, Hangzhou (CN)

(21) Appl. No.: 16/640,718

(22) PCT Filed: Dec. 24, 2018
(86) PCT No.: PCT/CN2018/123078

§ 371 Date: Feb. 20, 2020

(30) Foreign Application Priority Data
Apr. 20, 2018 (CN) 201810357705.7

(51) Int. Cl. A61N 1/372; A61N 1/378; H04B 5/00

(52) U.S. Cl. CPC A61N 1/37252; A61N 1/3787; H04B 5/0087; H04B 5/0037; H04B 5/0031

(57) ABSTRACT

The present invention relates to the field of medical appliances, more particularly to a communication system of an implantable device, comprising an external unit and an implantable unit, wherein the external unit and the implantable unit realize charging and bidirectional signal transmission of the external unit to the implantable unit by a wireless signal, two coupling coils are disposed outside the body, one for transmitting the forward signal, and one for receiving the reverse signal, and one coupling coil is disposed in the body for receiving and feeding back the signal, wherein the setting of the shape and position of the two coupling coils outside the body avoids the reverse signal fed back from inside of the body to be disturbed, thereby achieving effective transmission of the bidirectional signal, and overcoming the problem that the signal has weak signal strength and tends to be interfered when the traditional implantable device transmits the signal in a reverse direction.

### FIELD OF THE INVENTION

The present invention relates to the field of medical appliances, more particularly to a communication system of an implantable device and a communication method thereof.

### BACKGROUND OF THE INVENTION

An implant device generally uses coil coupling to transmit energy and signals, and traditional solutions generally use a structure of two coils, wherein one coil is disposed outside the body to transmit the signal, the other coil is disposed inside the body to receive the signal, and to archive charging of external to inside the body.

Since internal coupling coil L2 is restricted by size requirements, it is small in area and volume, while the size of the external coil L1 is not restricted by the installation, as a whole the coupling coefficient k12 is small, only by increasing the transmit power of L1, L2 can obtain more energy, so the voltage amplitude of the L2 coil is very large. After a forward signal is transmitted through L1 modulation, a signal with a certain envelop modulation depth (VPP_foward) is easily obtained on L2, this envelop depth fully satisfies demodulation requirements of the signal, thus forward communications can operate properly. Meanwhile, when sending reverse data transmission, there is a signal with amplitude modulation generates on the L2 coil, and a corresponding signal envelope (amplitude VPP_backward) appears on the L1 coil by load reflection, however, since k12 is very small, the amplitude of the useful signal envelope seen on L1 will be small. In some severe environments, the signal will be completely submerged by the signal and noise from L1, causing the receiver and demodulation unit in the external unit to fail to obtain the useful signal. Resulting in the reverse data work of the entire system failed.

Therefore, by simply using two coils is difficult to achieve effective transmission of forward and reverse data, and if the coupling coil of the reverse reception is further added in the external unit, it will be interfered by the coupling coil of the forward transmission signal, and effective transmission of the reverse signal is also difficult to achieve.

### BRIEF SUMMARY OF THE INVENTION

In order to solve the problem that reverse signal transmission work of traditional implanted devices is unfavorable, the present invention comprises two external coupling coils, wherein one is for transmitting the forward signal, and the other one is for receiving the reverse signal, and at the same time the shape and position setting of the two external coupling coils allows the reverse signal not to be interfered, thereby realizing the effective transmission of the bidirectional signal. The specific solutions are as follows:

A communication system of an implantable device, comprises an external unit and an implantable unit, wherein the external unit and the internal unit realize charging and bidirectional signal transmitting of the external unit to the implantable unit by a wireless signal, wherein the external unit comprises a digital signal processing unit, a power transmission unit and a receiver and demodulation unit, the internal unit comprises a stimulation module and a signal receiver and transmission unit, the power transmission unit comprises a first coupling coil L1, the first coupling coil is configured to transmit the signal to the internal unit, the signal receiver unit comprises a second coupling coil L2, the second coupling coil L2 is configured to receive the signal transmitted from the external unit and transmit a signal to the external unit, the receiver and modulation unit comprises a third coupling coil L3, and the third coupling coil is configured to receive the signal transmitted from the implantable unit.

Preferably, the third coupling coil L3 is disposed between the first coupling coil L1 and the second coupling coil L2.

Preferably, the center position of the first coupling coil L1 is aligned with the center position of the third coupling coil L3, and the third coupling coil L3 has a symmetrical shape, the magneto-inductive currents of the first coupling coil L1 coupled through the third coupling coil L3 are canceled each other out.

Preferably, the size of the first coupling coil L1 is larger than the size of the second coupling coil L2, and the projection of the second coupling coil L2 and the projection of the first coupling coil L1 overlap.

Preferably, the second coupling coil L2 is in the projection of the first coupling coil L1 and the third coupling coil L3.

Preferably, the third coupling coil L3 comprises two symmetrical parts, the coils on the two symmetrical parts are arranged to cross each other through a symmetric center, the cross point thereof overlaps with the center of the first coupling coil, there is an offset between the center of the second coupling coil L2 and the center cross point position of the third coupling coil L3, and positive and negative currents generated on the third coupling coil are canceled each other out, when the first coupling coil L1 is coupled.

Preferably, the first coupling coil is a toroidal coil, and the two symmetrical parts of the third coupling coil are both loop coils with a curvature.

Preferably, the first coupling coil is a square loop coil, and the two symmetrical parts of the third coupling coil are both square loop coils.

Preferably, the third coupling coil L3 comprises a region S1 overlapping with the projection of the first coupling coil L1 and a region S2 not overlapping with the projection of the first coupling coil, and when the first coupling coil L1 is coupled, positive and negative magnetic fluxes passing through two regions of the third coupling coil L3 are canceled each other out.

Preferably, a signal shielding device for preventing interference with communications among the first coupling coil L1, the second coupling coil L2 and the third coupling coil L3 is further disposed outside the first coupling coil L1.

Beneficial effects of the present invention: two coupling coils are disposed outside the body, wherein one is for transmitting the forward signal, and the other one is for receiving the reverse signal, while the setting of the shape and position of the two coupling coils outside the body, are such that the reverse signal is not disturbed, thereby achieving effective transmission of the bidirectional signal. The problem that the signal has weak signal strength and tends to be interfered when the traditional implantable device transmits the signal in a reverse direction is overcome.

### BRIEF DESCRIPTION OF THE DRAWINGS

FIG. 1 is a communication schematic view of a traditional communication system;

FIG. 2 is a communication waveform diagram of a traditional communication system;

FIG. 3 is a communication schematic view of a communication system of the present intention;

FIG. 4 is a communication waveform diagram of a communication system of the present invention;

FIG. 5 is a schematic view showing the position and shape of a coil according to Example One of the present invention;

FIG. 6 is a schematic view showing the position and shape of a coil according to Example Two of the present invention;

FIG. 7 is a schematic view showing the position and shape of a coil according to Example Three of the present invention.

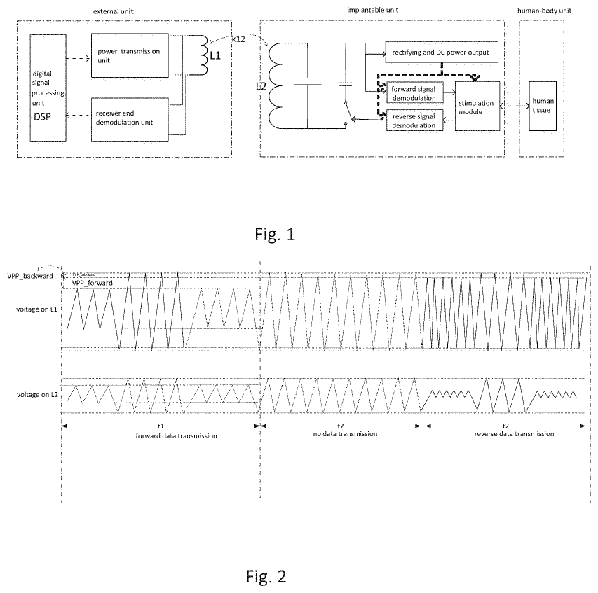

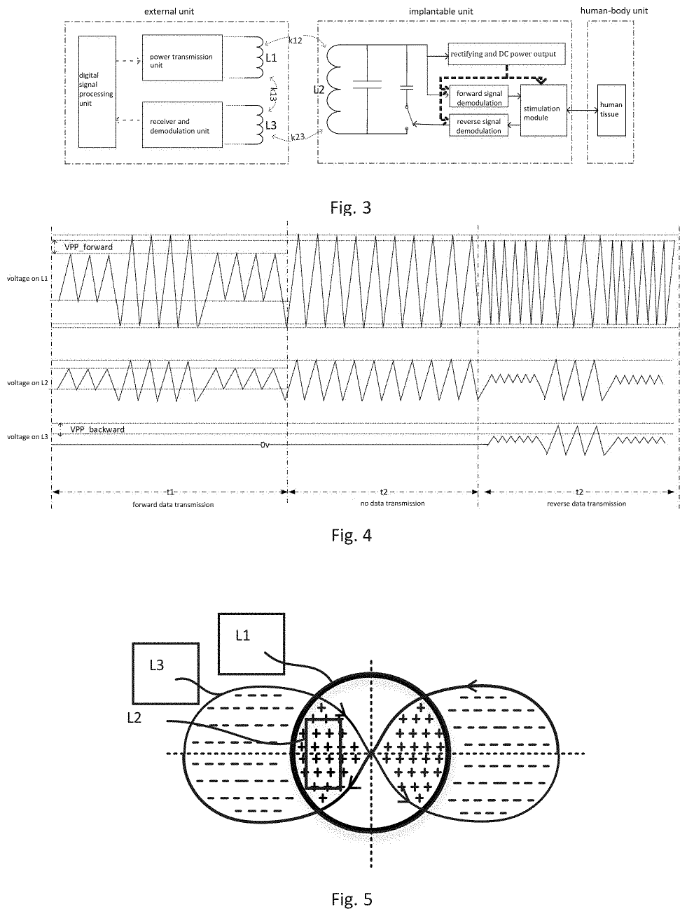

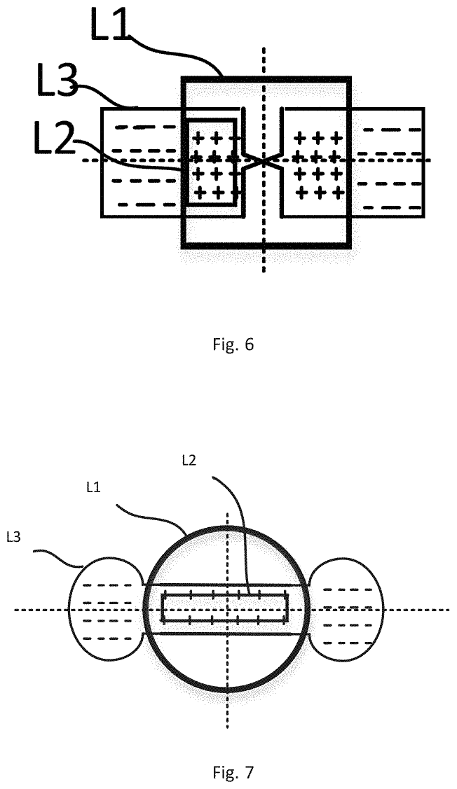

### DETAILED DESCRIPTION OF EMBODIMENTS

In order to facilitate a further understanding of the present invention, it will be further illustrated below with reference to the accompanying drawings.

As shown in the drawings, the present invention firstly discloses a communication system of an implantable device, comprising: an external unit and an implantable unit, wherein the external unit and the implantable unit realize charging and bidirectional signal transmitting of the external unit to the implantable unit by a wireless signal. In which, the external unit comprises a digital signal processing unit, a power transmission unit and a receiver and demodulation unit, and the internal unit comprises a stimulation module and a signal receiver and transmission unit. The power transmission unit comprises a first coupling coil L1, the first coupling coil is configured to transmit a signal to the internal unit, the signal receiver unit comprises a second coupling coil L2, and the second coupling coil L2 is configured to receive the signal transmitted from the external unit and transmit a signal to the external unit, the receiver and modulation unit comprises a third coupling coil L3, and the third coupling coil is configured to receive the signal transmitted from the implantable unit. The third coupling coil L3 is disposed between the first coupling coil L1 and the second coupling coil L2.

During the signal transmission, the external unit transmits a signal to the internal unit via coupling between the first coupling coil and the second coupling coil in the internal unit, and the internal unit receives the signal and achieves charging.

Since the size of the second coupling coil L2 in the internal unit is generally small, in order for the internal unit to obtain more energy, it is necessary to increase the transmission power of the external unit, and it is necessary to increase the size of the first coupling coil in the external unit, and increase the coupling coefficient k12. If the first coupling coil is continuously used to receive the reverse signal transmitted from the internal unit, the reverse coupling coefficient k21 will be small, when the reverse data transmission is transmitted, there is a signal with an amplitude modulation on the second coupling coil, and a corresponding signal envelope (amplitude VPP_backward) appears on the L1 coil by load reflection, however, since coupling coefficient k21 is very small, the envelop amplitude of useful signal seen on the L1 will be very small, and in some severe environments, the signal will be completely submerged by the signal and the noise from L1, causing the receiver and demodulation unit in the external unit to fail to receive the useful signal, resulting in failure of the reverse data work of the entire system.

The present invention uses the third coupling coil L3 to receive the reverse signal, during the signal transmission, the center position of the first coupling coil L1 is aligned with the center position of the third coupling coil L3, and the third coupling coil L3 has a symmetrical shape, so that magnetic induction currents of the first coupling coil L1 coupled through the third coupling coil L3 are canceled each other out.

In general, the size of the first coupling coil L1 is larger than the size of the second coupling coil L2, and the projection of the second coupling coil L2 and the projection of the first coupling coil L1 overlap, ensuring the signal forwardly transmitted by the first coupling coil L1 will be effectively received by the second coupling coil L2 disposed in the body. Furthermore, the second coupling coil L2 is in the projection of the first coupling coil L1 and the third coupling coil L3, the coupling effect of the first coupling coil L1 and the second coupling coil L2 is better.

In addition, a signal shielding device for preventing interference with communications among the first coupling coil L1, the second coupling coil L2 and the third coupling coil L3 is further disposed outside the first coupling coil L1, and such signal shielding device can make the communications among the coupling coils more stable.

In order to prevent the reverse signal transmission from being affected by the first coupling coil and the second coupling coil, the position and shape setting of the third coupling coil can be implemented in various embodiments, and several are listed below.

### Example 1

The third coupling coil L3 comprises two symmetrical parts, the coils on the two symmetrical parts are arranged to cross each other through a symmetric center, its cross point overlaps with the center of the first coupling coil, there is an offset between the center cross point position of the second coupling coil L2 and the third coupling coil L3, and positive and negative currents generated on the third coupling coil are canceled each other out, when the first coupling coil is coupled, and when the second coupling coil is coupled, the third coupling coil can fully receive the signal. In this Example, the first coupling coil is a toroidal coil, and the two symmetrical parts of the third coupling coil are both loop coils with a curvature.

### Example 2

The third coupling coil L3 comprises two symmetrical parts, the coils on the two symmetrical parts are arranged to cross each other through a symmetric center, its cross point overlaps with the center of the first coupling coil, there is an offset between the center cross point position of the second coupling coil L2 and the third coupling coil L3, and positive and negative currents generated on the third coupling coil are canceled each other out, when the first coupling coil is coupled, and when the second coupling coil is coupled, the third coupling coil can fully receive the signal. The first coupling coil is a square loop coil, and the two symmetrical parts of the third coupling coil are both square loop coils.

### Example 3

The third coupling coil L3 comprises a region S1 overlapping with the projection of the first coupling coil L1 and a region S2 not overlapping with the projection of the first coupling coil L1, through detecting and testing, a position of the third coupling coil relative to the first coupling coil is properly placed, so that the positive and negative magnetic fluxes passing through two regions of the third coupling coil L3 are canceled each other out, when the first coupling coil L1 is coupled. The third coupling coil L3 comprises an intermediate portion coil having a “one” word shape and the end coil connected to the two ends of the intermediate portion, wherein the intermediate portion coil overlaps with the first coupling coil, and the two end coils does not overlap with the first coupling coil. When the signal is transmitted in a reverse direction, the positive and negative magnetic fluxes passing through the overlap region S1 and the non-overlap region S2 of the third coupling coil are equal, therefore when the signal is reversely transmitted, the coupling of the second coupling coil and the first coupling coil does not cause interference to the signal received by the third coupling coil.

The aforementioned examples are merely preferred examples of the present invention, which are not intended to limit the present invention. It should be understood that, any modifications, equivalent replacements, improvements, etc. made according to the spirit and principles of the present invention are within the protection scope of the invention.

What is claimed is:

1. A communication system of an implantable device, characterized in that, comprises an external unit and an implantable unit, and the external unit and the implantable unit realize charging and bidirectional signal transmission of the external unit to the implantable unit by a wireless signal, wherein the external unit comprises a digital signal processing unit, a power transmission unit and a receiver and demodulation unit, the internal unit comprises a stimulation module and a signal receiver and transmission unit, the power transmission unit comprises a first coupling coil L1, the first coupling coil is configured to transmit a signal to the internal unit, the signal receiver unit comprises a second coupling coil L2, the second coupling coil L2 is configured to receive the signal from the external unit and transmit a signal to the external unit, the receiver and modulation unit comprises a third coupling coil L3, and the third coupling coil is configured to receive the signal from the implantable unit.

2. The communication system of the implantable device according to claim 1, characterized in that, the third coupling coil L3 is disposed between the first coupling coil L1 and the second coupling coil L2.

3. The communication system of the implantable device according to claim 1, characterized in that, a center position of the first coupling coil L1 is aligned with a center position of the third coupling coil L3, and the third coupling coil L3 has a symmetrical shape, the magnetic induction current of the first coupling coil L1 coupled through the third coupling coil L3 are canceled each other out.

4. The communication system of the implantable device according to claim 3, characterized in that, a size of the first coupling coil L1 is larger than a size of the second coupling coil L2, and the projections of the second coupling coil L2 and the first coupling coil L1 overlap.

5. The communication system of the implantable device according to claim 4, characterized in that, the second coupling coil L2 is in the projections of the first coupling coil L1 and the third coupling coil L3.

6. The communication system of the implantable device according to claim 1, characterized in that, the third coupling coil L3 comprises two symmetrical parts, the coils on the two symmetrical parts are arranged to cross each other through a symmetric center, a cross point thereof overlaps with a center of the first coupling coil, there is an offset between a center of the second coupling coil L2 and a center cross point position of the third coupling coil L3, and a positive and negative current generated on the third coupling coil are canceled each other out, when the first coupling coil L1 is coupled.

7. The communication system of the implantable device according to claim 6, characterized in that, the first coupling coil is a toroidal coil, and the two symmetrical parts of the third coupling coil are both loop coils with a curvature.

8. The communication system of the implantable device according to claim 6, characterized in that, the first coupling coil is a square loop coil, and the two symmetrical parts of the third coupling coil are both square loop coils.

9. The communication system of the implantable device according to claim 1, characterized in that, the third coupling coil L3 comprises a region S1 overlapping with the projection of the first coupling coil L1 and a region S2 not overlapping with the projection of the first coupling coil L1, and a positive and negative magnetic flux passing through two regions of the third coupling coil L3 are canceled each other out, when the first coupling coil is coupled.

10. The communication system of the implantable device according to claim 1, characterized in that, a signal shielding device is further disposed outside the first coupling coil L1 for preventing interference with communications among the first coupling coil L1, the second coupling coil L2 and the third coupling coil L3.
